(19) United States
(12) Patent Application Publication
(10) Pub. No.: US 2026/0174637 A1
(43) Pub. Date: Jun. 25, 2026

(54) PERSONAL CARE APPARATUS AND PERSONAL CARE ROOM COMPRISING THE SAME

(71) Applicant: Shota KOIZUMI, Setagaya-ku, Tokyo (JP)

(72) Inventor: Shota KOIZUMI, Setagaya-ku, Tokyo (JP)

(21) Appl. No.: 19/126,861

(22) PCT Filed: Nov. 6, 2023
(86) PCT No.: PCT/JP2023/039920

§ 371 Date: May 2, 2025

(30) Foreign Application Priority Data
Nov. 4, 2022 (JP) 2022-177447

(51) Int. Cl. A61K 8/02; A47L 13/22

(52) U.S. Cl. CPC A61K 8/02; A47L 13/225

(57) ABSTRACT

An object of the present disclosure is to improve the efficacy that is directly acted on a body through an approach that has not been available in conventional apparatuses or facilities, and allow users to easily increase their deep body temperature, thereby contributing to improving people's immunity and healing ability against diseases. In order to achieve such object, a personal care apparatus according to the present disclosure comprises a bedrock 16RP containing lava that has air permeability and generates far-infrared radiation, an electric heater that heats the bedrock 16RP, a plate-shaped heat insulating material, a plate-shaped holding member that supports a back surface of the heat insulating material, a metal film member, a temperature sensor mounted to the bedrock 16RP, a temperature regulating part that regulates the amount of heat generated by the electric heater, a control apparatus that controls a surface temperature of the bedrock 16RP to be 38-43° C., an audio signal transmission system 81 that transmits an audio signal to the user, and an audio output system 82 that outputs sound, wherein each of the bedrock, the heater and the heat insulating material is simply placed, without being fixed, on an upper side of the holding member.

### TECHNICAL FIELD

The present disclosure relates to a personal care apparatus and a personal care room comprising such personal care apparatus.

### BACKGROUND ART

Facilities, such as Japanese tearooms, in which users engage in meditation to face and calm themselves, and facilities for conditioning one's physical state or autonomic nervous system through yoga or stretching exercises, have been widely utilized as facilities for alleviating fatigue or stress, providing relaxing and healing effects, and thereby providing physical and mental care and helping users to lead healthy lives. In this specification, an apparatus that constitutes or is utilized in the facilities having such purposes will be referred to as a “care apparatus,” and, in particular, an apparatus that is personally utilized will be referred to as a “personal care apparatus” (see, for example, patent literature 1). In addition, a facility or room that comprises such personal care apparatus and is intended for one person or a few persons will be referred to as a “personal care room.”

By using, for example, such apparatus (personal care apparatus) or facility (personal care room) to condition one's physical state or autonomic nervous system, alleviate fatigue and stress, and providing physical and mental care through relaxing and healing effects, will lead to improved immunity of a body. However, in recent years, the average body temperature of Japanese people has become lower than before and, based on recently obtained findings, such lowering of the body temperature has weakened the immunity and the ability to heal against disease. For example, although there are varieties of therapies available against cancers, there are reports indicating that the healing ability has improved by increasing the body temperature by 1° C. and without relying on administering drugs. It should be noted, however, that this does not mean that it is sufficient to simply increase a body surface temperature, and the above-described effect can instead be achieved by appropriately increasing a deep body temperature. The deep body temperature refers to a temperature in an organ inside a body, and among all of these, it is considered that a rectal temperature is particularly important in relation to the above-described immunity and healing ability.

One easy way to increase the body temperature may be through utilizing saunas. In particular, saunas have become popular in recent years, and it can certainly be said that it is healthy to casually access a sauna as a leisure activity or for recreational purposes, and to relax both physically and mentally while increasing the body temperature to condition one's physical state. However, when it comes to saunas, which, as stated above, have become increasingly popular in recent years and seem to have gained an increasing number of enthusiasts, it is not difficult to imagine that there is a certain percentage of users who cannot completely enjoy the comfort without feeling a certain degree of discomfort while using saunas. In fact, the present inventor is also one of those users, and the inventor feels that saunas can be a little painful, or there can be an experience of a feeling of pressure where the inventor has to endure a little longer in such a hot room in order to sufficiently sweat or increase the body temperature. More specifically, a sauna requires a user to stay in a room for several minutes where the room temperature is set to, for example, 90-100° C. and the humidity is set to around 20%, in order to cause the user to sweat a lot. Furthermore, although such situation is true in the case of “Finnish saunas,” which are said to have a relatively mild indoor environment, in the case of so-called dry-type saunas, a user has to stay within a harsh environment where the room temperature is set to 80-90° C. and the humidity is set to extremely low, at around a few percent, which may damage the mucous membranes of the nose and throat, as well as the airways and lungs, and may involve severe physical load in order for the user to sweat in such hot and dry conditions. For enthusiasts, remaining in such environment may even be the key to achieving an extraordinary sense of euphoria in a subsequent cold bath (alternating between hot and cold baths) or an open-air bath; however, for users who have not yet reached that level, especially for beginners, such harsh environment may be a high hurdle to overcome. Therefore, it is difficult to consider that conventional sauna facilities are suitable for increasing the deep body temperature (especially the rectal temperature).

Under such background and circumstances, it is believed that facilities other than saunas (such as personal care rooms) that are useful for increasing the deep body temperature (especially rectal temperature) could contribute to improving immunity and healing ability against diseases for many people. This is an objective that the inventor considers as a mission to be achieved.

### CITATION LIST

### Patent Literature

Patent Literature 1: JP2020-500679 A

### SUMMARY OF INVENTION

### Technical Problem

However, there have in fact not been any revolutionary personal care apparatuses or personal care rooms that have been proposed in order to directly address the above-described objective (mission). If such personal care apparatuses, etc. are equipped with a mechanism that can take advantage of their characteristics while further enhancing their efficacy, it is considered possible to provide unique additional value that cannot be obtained from other apparatuses.

In view of the above, an object of the present disclosure is to provide a personal care apparatus, as well as a personal care room comprising such personal care apparatus, that are capable of improving the efficacy that is directly acted on a body through an approach that has not been available in conventional apparatuses or facilities, allowing users to easily increase their deep body temperature, and thereby contributing to improving their immunity and healing abilities against diseases.

### Solution to Problem

In order to achieve the object above, the inventor has repeatedly conducted various studies and tests, and achieved a certain finding based on an idea to create a new mechanism that provides a comfort which has never been experienced before, heats a body from its core to thereby exert an effect on a deep part of the body, and utilizes such characteristics to further enhance the efficacy of the apparatus.

A personal care apparatus according to the present disclosure based on the above-described finding is a personal care apparatus constituting a personal care room that can be used to increase a deep body temperature of a user, the apparatus comprising: a bedrock installed on a floor such that a proportion of the bedrock occupying a surface area inside the personal care room is 10% or more, the bedrock containing lava that has air permeability and generates far-infrared radiation; an electric heater that heats the bedrock from an inside or a backside thereof; a plate-shaped heat insulating material arranged so as to support a bottom surface of the electric heater from below; a plate-shaped holding member arranged so as to support a back surface of the heat insulating material; a metal film member provided at least on an upper surface of the electric heater; a temperature sensor mounted in a recess formed in a front surface of the bedrock; a temperature regulating part that regulates a magnitude of electric power to be supplied to the electric heater so as to regulate an amount of heat generated by the electric heater; a control apparatus that controls a surface temperature of the bedrock to be 38-43° C.; an audio signal transmission system that transmits an audio signal to the user; and an audio output system that outputs sound based on the audio signal transmitted from the audio signal transmission system, wherein each of the bedrock, the heater and the heat insulating material is simply placed, without being fixed, on an upper side of the holding member.

Such personal care apparatus utilizes far-infrared radiation heat to thereby make it possible to improve a physical sensation and an effect during physical and mental care to higher levels than those provided by conventional apparatuses, and to also enable a thermal approach to a deep part of a body due to the particular effect of the far-infrared radiation heat and consequently accelerate a high metabolic activity, whereby it can be considered that the personal care apparatus comprises a configuration that is necessary to achieve unique effects that have not been easily achievable with the conventional personal care apparatuses. In other words, it can be considered that such personal care apparatus synergistically provides the effects that are directly acted on the body not only from mental and internal perspectives, but also from physical and external perspectives based more on medical evidence. Such apparatus makes it possible to easily increase the user's deep body temperature (particularly the rectal temperature).

Furthermore, such personal care apparatus enables the user to not only easily enjoy the effects of far-infrared radiation heat while relaxing by lying down or sitting on the floor and engaging in meditation, but also to obtain synergistic effects by engaging in a mild workout or gymnastics such as stretching, yoga, or exercises in the room while enjoying the-above-described effects. In this regard, according to the personal care apparatus of the present disclosure, which comprises an audio signal transmission system and an audio output system, it is possible to not only provide the user with healing music and the like, but also to play sound for prompting a mild workout or gymnastics (for example, a pre-recorded guide sound that serves as a guidance for the workout or gymnastics) to thereby encourage the user to engage in such workout or gymnastics from the user's own motivation or to utilize such sound as an invitation to perform a workout or gymnastics, which makes it possible to construct a unique apparatus that can help the user to effectively increase his/her deep body temperature.

The personal care apparatus described above may further comprise a metal panel member arranged between the metal film member and the bedrock.

In the personal care apparatus described above, the audio output system may comprise wireless earphones worn by the user.

In the personal care apparatus described above, the bedrock installed on the floor may have a size and a shape that are sufficient for the user to take a supine or prone position.

The personal care apparatus described above may further comprise a steam generating apparatus that supplies steam inside the personal care room.

In the personal care apparatus described above, the steam generating apparatus may boil water or hot water with herbal medicine therein and supply steam containing an herbal medicine component.

In the personal care apparatus described above, a medicinal herb having a drug efficacy or aroma may be used as the herbal medicine.

In the personal care apparatus described above, the medicinal herb may have been pulverized and packed in a bag.

In the personal care apparatus described above, the steam generating apparatus may be provided in the personal care room.

The personal care apparatus described above may further comprise an air blowing apparatus that blows air so as to cause the steam containing the herbal medicine component supplied from the steam generating apparatus to move toward an upper portion of the room and then cause the steam to move downward from the upper portion of the room due to air circulation inside the room.

In the personal care apparatus described above, the control apparatus may control a room temperature inside the personal care room to be 35-40° C. and control a humidity to be 60% or higher.

A personal care room according to the present disclosure comprises the personal care apparatus described above.

The personal care room described above may be formed in a size and a shape intended for a single user, or may be formed in a size and a shape sufficient for one or multiple users to occupy and engage in exercise therein.

### BRIEF DESCRIPTION OF DRAWINGS

FIG. 1 is a plan view of the entire personal care room, which shows an example of a personal care apparatus.

FIG. 2 is a diagram taken along line II-II in FIG. 1, showing an example of an interior of the personal care room during use.

FIG. 3 is a conceptual diagram showing an example of the entire personal care room as viewed from the front.

FIG. 4 is a conceptual diagram showing the interior of the personal care room shown in FIG. 3.

FIG. 5 is a diagram schematically showing a configuration example of a heating apparatus that constitutes the personal care apparatus.

FIG. 6 is a diagram showing a user engaging in exercise while listening to sound from an audio output system in the personal care room.

### DESCRIPTION OF EMBODIMENTS

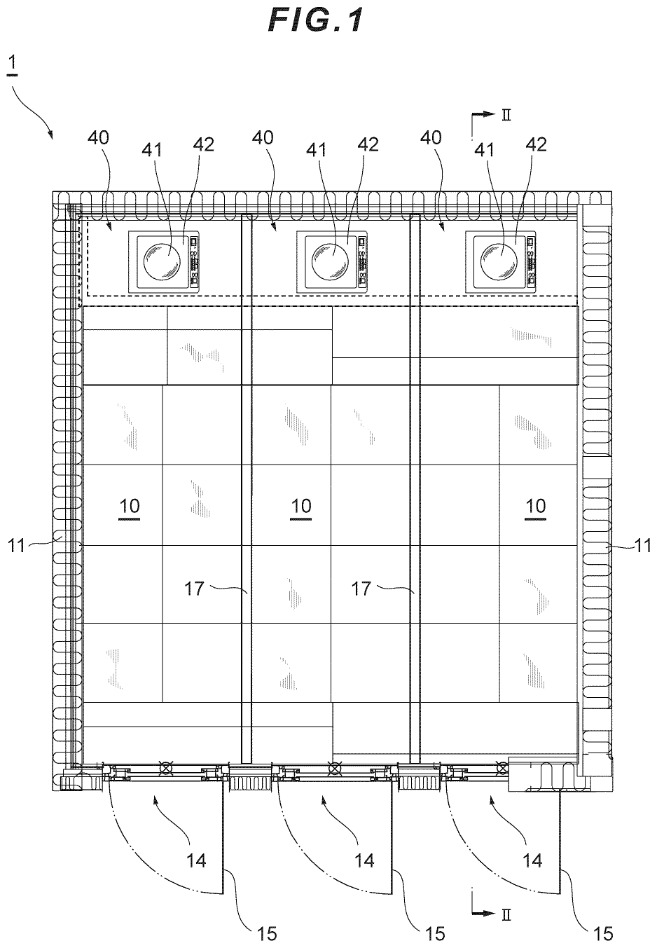

Preferred embodiments of a personal care apparatus and a personal care room according to the present disclosure will now be described in detail below while referring to the attached drawings (see FIG. 1, etc.).

### Configuration of Personal Care Apparatus

A personal care apparatus 1 according to the present embodiment comprises an interior material 16, a heating apparatus 30, a steam generating apparatus 40, an air blowing apparatus 50, a control apparatus 70, etc., which are installed inside a personal care room 10.

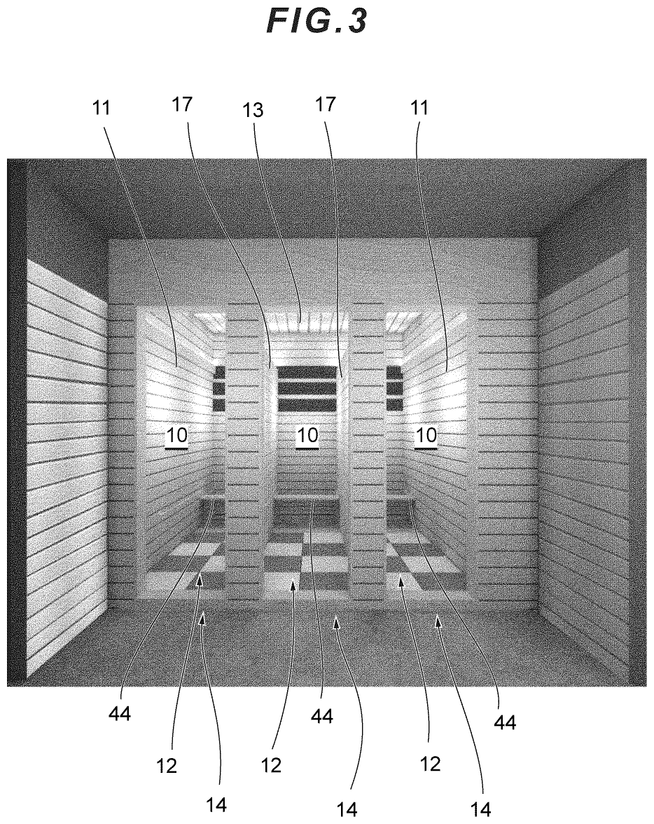

The size or configuration of the personal care room 10 is not particularly limited, and it may be sized or configured so as to be used only by a single user (alone) or it may alternatively be sized or configured so that more than one user can enter at one time. The personal care room 10 according to the present embodiment which shows a preferred example has a size and a shape sufficient for a single user to engage in mild exercise in a supine or prone state, a seated state or a standing state, and, for example, three of such personal care rooms 10 may be arranged side-by-side (see FIGS. 1, 3, etc.).

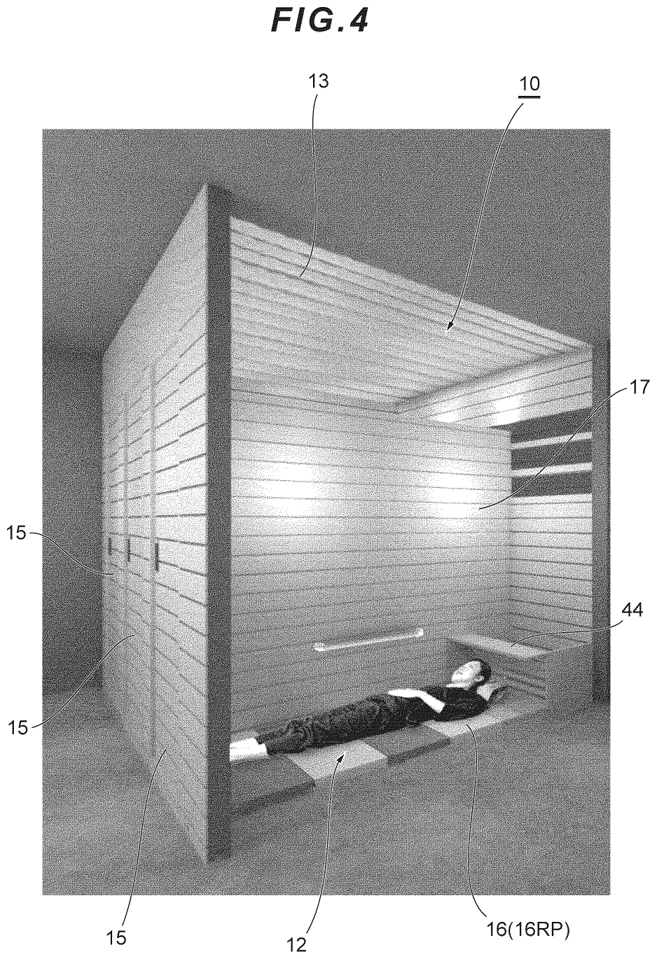

Each of such personal care rooms 10 includes a wall 11, a floor 12, a ceiling 13, an entrance/exit 14, a door 15, the interior material 16, a partition wall 17, etc. (see FIG. 1, etc.). The three personal care rooms 10 are partitioned by the partition walls 17 having only upper portions thereof opened so that adjacent rooms communicate with each other (see FIG. 3, etc.). Each of the three personal care rooms 10 is provided with the entrance/exit 14 having the door 15 (see FIG. 1). A system for helping a user to stay more comfortably or a system for teaching exercise, such as a receiving set (not shown), a speaker (not shown) and an indirect lighting apparatus 60, may also be provided inside the personal care room 10 (see FIGS. 1 and 4).

In the interior material 16 constituting the personal care room 10, a bedrock 16RP that contains ores is arranged in a portion of the floor 12 (see FIG. 4, etc.). Although the bedrock 16RP may be installed in a portion occupying the entire floor 12, the bedrock 16RP may be installed in various manners such as, for example, arranging it only in a portion of the floor 12 or further arranging it in a portion of the wall 11 or the partition wall 17 (although this is not particularly shown). The present embodiment employs, as the bedrock 16RP, a bedrock containing an ore which generates far-infrared radiation, more specifically, a bedrock containing lava or a predetermined ore. In general, the bedrock 16RP has air permeability. It should be noted that the term “a predetermined ore” as used herein refers to an ore having a high heat radiation effect for far-infrared radiation, such as, for example, a basalt rock.

The use of the bedrock 16RP in a portion (mainly in the floor 12 in the case of the present embodiment) of the interior of the personal care room 10 has the following advantages, which are commonly known.
The far-infrared radiation effect provided by the bedrock 16RP indirectly heats a body, which causes an increase in the body temperature, in particular, the deep body temperature such as the rectal temperature. In general, it is said that a low body temperature causes various types of poor health conditions (such as infection by cold viruses, allergic symptoms, indigestion, constipation, chronic fatigue, damaged skin, acne, cold constitution, irregular menstruation, infertility, decrease in sleeping quality, shoulder stiffness, backache, gray hair, and hair loss), and the bedrock 16RP is effective in preventing or improving these poor health conditions.
It is said that the thermal effect of the far-infrared radiation continues for about 72 hours and, as can be seen from the fact that the far-infrared radiation is called “heat radiation,” it steadily heats a body from the inside thereof, which promotes blood circulation and enhances metabolism. The promotion of blood circulation also has the effects of achieving recovery from fatigue and improving hepatic functions and cold constitution.
When a body is heated from the inside thereof, the body is caused to sweat from the sebaceous glands. Profuse sweating is expected to provide a so-called detoxification effect wherein toxic substances and waste products are excreted from the body. At this time, skin metabolism is also accelerated, which is expected to provide cosmetic effects such as resolving skin problems and achieving beautiful skin.
In use, the bedrock 16RP is heated to thereby cause minerals contained in the lava, etc. to be released in the air. Vanadium contained in the minerals is considered to have an effect of reducing blood pressure, blood glucose level and cholesterol, and thus have an effect of preventing lifestyle-related disorders.
Negative ions emitted from the lava, etc. are also considered to have a relaxing effect.

One of the advantages provided by the use of the bedrock 16RP in a portion (mainly in the floor 12 in the case of the present embodiment) of the interior of the personal care room 10 is so-called “earthing,” which will be described below.
Earthing refers to an act of a person directly touching the ground, etc. with his/her bare feet by taking off his/her shoes and socks to discharge electricity that has resided in the body, or to a health regimen using such method. Since earthing involves connection with the ground, it is also called “grounding.” Since a human body consists mainly of water, it easily conducts electricity, and while living in today's society where people are surrounded by electromagnetic waves from electric appliances and mobile phones, their bodies may be electrically charged without realizing it. Certain opinions have been presented which argue that the above-described situation could be one of the causes for modern diseases, such as headache, shoulder stiffness and poor blood circulation, from which many people are suffering nowadays. It is said that our predecessors discharged electricity that accumulated inside their bodies and maintained a good balance by interacting with nature; however, modern people cannot experience such situation, because they walk on concrete while wearing shoes with rubber soles. Under such circumstances, there has been a proposal which encourages people to touch the grounds with their bare skin to thereby discharge the electricity within their bodies so as to maintain a good balance of the electrical energy within their bodies, thereby maintaining their physical conditions. Such approach enables recovery from fatigue, alleviation of inflammation or pain, mental stabilization, improvement of sleeping quality, lowering of high blood pressure, improvement of blood circulation, conditioning of the autonomic nervous system, improvement of hormonal imbalance, and so on. As in the present embodiment, the bedrock 16RP containing an ore that generates far-infrared radiation, more specifically, the bedrock 16RP containing lava or a predetermined ore, etc., has the earthing effect described above.

Although the bedrock 16RP does not have to be installed in all of the wall 11, the floor 12, the ceiling 13, and the partitioning wall 17 of the personal care room 10, in order for various effects and the above-described advantages that can be experienced inside the personal care room 10 to be exerted more effectively, the proportion of the bedrock 16RP that occupies the surface area inside the personal care room 10 is preferably a predetermined value or more, e.g., 10% or more, and more preferably 15% or more. In the present embodiment, in consideration of the various effects and efficacies, the floor 12 is made of the bedrock 16RP containing lava or a predetermined ore, so that such bedrock 16RP occupies 10% or more of the surface area inside the personal care room 10. In this way, in the personal care apparatus 1 of the present embodiment in which the floor 12 is made of the bedrock 16RP, the area of the bedrock 16RP which the user who is in a supine or prone position contacts directly or indirectly via his/her clothes in the personal care room 10 is relatively large (see FIG. 4, etc.). The thickness of the bedrock 16RP may be, for example, 2 to 5 cm.

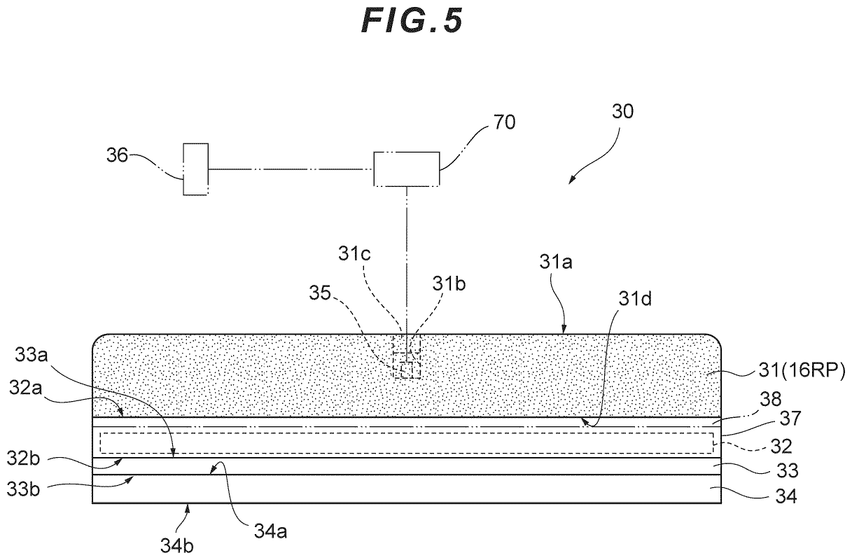

The heating apparatus 30 is an apparatus for heating the body of a user who is in a supine or prone position or a user in a seated position or a standing position on the bedrock 16RP. Referring now to FIG. 5, the configuration of the heating apparatus 30 will be described below. FIG. 5 shows the heating apparatus 30 as viewed from the front. The heating apparatus 30 includes a supporting member 31, a heater 32, a heat insulating material 33, a holding member 34, an aluminum foil member 37, and a stainless-steel panel 38.

The supporting member 31 is a member which is arranged at a top of the heating apparatus 30. The supporting member 31 has a rectangular shape when viewed from above, and supports the body of the user who is in a supine or prone position on a front surface 31a of the supporting member 31. In the present embodiment, the supporting member 31 is constituted by the bedrock 16RP.

A recess 31b is formed at a central position of the front surface 31a of the supporting member 31. The depth of the recess 31b may be, for example, one half (½) of the thickness of the supporting member 31. A temperature sensor 35 is mounted on a bottom surface of the recess 31b. The temperature sensor 35 is a sensor for measuring the temperature of the supporting member 31, which may be, for example, a thermistor. A signal indicating the temperature measured by the temperature sensor 35 is input to the control apparatus 70. A portion of the recess 31b which is located above the temperature sensor 35 is closed by a lid 31c.

The heater 32 is an electric heater that heats the supporting member 31 from a back surface 31d thereof. The shape of the heater 32 as viewed from above is the same as the shape of the supporting member 31. The heater 32 is constituted as a rubber heater made of silicone. The heater 32 is arranged such that the entire front surface 32a thereof is brought into close contact with the back surface 31d of the supporting member 31. However, the supporting member 31 and the heater 32 are not fixed to each other. In other words, the supporting member 31 is merely placed from above on the front surface 32a of the heater 32.

When electric power is supplied from an external power supply (not shown) to the heater 32, the heater 32 generates heat, which heats the supporting member 31. The magnitude of the electric power to be supplied to the heater 32 is regulated by the control apparatus 70 and a temperature regulating part 36 (described later).

The heat insulating material 33 is a plate-shaped member that is arranged so as to support a back surface 32b of the heater 32 from below. That is to say, the heat insulating material 33 is arranged at a position opposite to the supporting member 31 with the heater 32 therebetween. The shape of the heat insulating material 33 as viewed from above is the same as the shape of the heater 32. The heat insulating material 33 is formed of calcium silicate. Although the material of the heat insulating material 33 may contain only calcium silicate as in the present embodiment, it may also contain other components in addition to the calcium silicate. Alternatively, the heat insulating material 33 may be formed of a material that does not contain calcium silicate, as long as such material can secure heat insulation capacity equal to or greater than that of the calcium silicate. The heat insulating material 33 of the present embodiment is arranged such that the entire front surface 33a thereof abuts on the back surface 32b of the heater 32. However, the heater 32 and the heat insulating material 33 are not fixed to each other. In other words, the heater 32 is merely placed from above on the front surface 33a of the heat insulating material 33.

The holding member 34 is a plate-shaped member arranged so as to support a back surface 33b of the heat insulating material 33. The shape of the holding member 34 as viewed from above is the same as the shape of the heat insulating material 33. The holding member 34 is formed of plywood. The holding member 34 is arranged such that the entire front surface 34a thereof abuts on the back surface 33b of the heat insulating material 33. However, the heat insulating material 33 and the holding member 34 are not fixed to each other. In other words, the heat insulating material 33 is merely placed from above on the front surface 34a of the holding member 34. The holding member 34 of the present embodiment holds all parts of the supporting member 31, the heater 32 and the heat insulating material 33 from below, and such configuration enhances the strength of the heating apparatus 30. A back surface 34b of the holding member 34 is fixed to a frame (not shown) that is provided on the inner side of the floor 12 of the personal care room 10. Therefore, the holding member 34 cannot be removed from the floor 12. On the other hand, each of the supporting member 31, the heater 32 and the heat insulating material 33 is merely placed from above on the holding member 34, without being fixed thereto, as described above, and is therefore removable from the holding member 34.

The temperature regulating part 36 is an apparatus that regulates the temperature of the supporting member 31 to a predetermined target temperature. The temperature regulating part 36 regulates the magnitude of the electric power to be supplied to the heater 32 to thereby regulate the amount of heat generated by the heater 32, and inputs to the control apparatus 70 a signal for causing the temperature of the supporting member 31 that is measured by the temperature sensor 35 to match with the target temperature described above. The temperature regulating part 36 may be configured as an input portion which the user, etc. manipulates in order to set the target temperature. In such case, the control apparatus 70 may have a function as a remote controller manipulated by the user. The control apparatus 70 may be configured as a wired remote controller or a wireless remote controller. When manipulated by the user, the temperature regulating part 36 sets the target temperature based on such manipulation.

The aluminum foil member 37 is an example of a metal film member provided at least on an upper surface of the heater 32. The aluminum foil member 37 of the present embodiment is provided so as to cover the heater 32. By providing such aluminum foil member (metal film member) 37 at least on the upper surface of the heater 32, the heat from the heater 32 can be transferred evenly onto the supporting member 31, which makes it possible to uniformly heat the supporting member 31.

The stainless-steel panel 38 is an example of a metal panel member which is provided as needed between the aluminum foil member 37 and the supporting member 31. The provision of such stainless-steel panel (metal panel member) 38 makes it possible to increase the strength of the apparatus without affecting the heat transfer performance thereof.

The steam generating apparatus 40 is an apparatus that is provided for supplying steam containing an herbal medicine component into the personal care room 10. According to the personal care apparatus 1 comprising such steam generating apparatus 40 of the present embodiment, the user's body is heated in the room so as to increase his/her sweating power and accelerate his/her metabolism, and, in such condition, the steam containing the herbal medicine component is supplied, which can create an environment where the user can easily take the herbal medicine component into his/her body through his/her respiratory organs and skin without feeling pain.

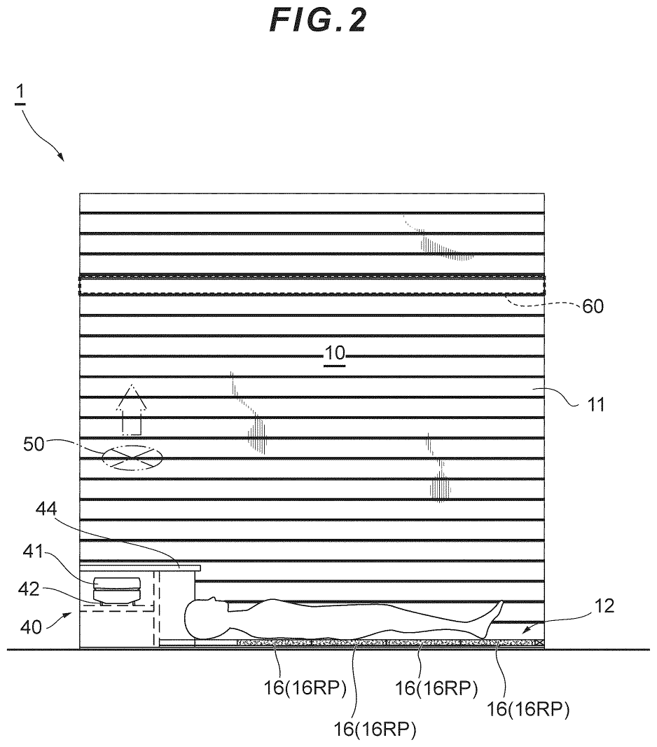

The present embodiment employs the steam generating apparatus 40 comprising a pot 41 for boiling water or hot water with an herbal medicine therein (see FIG. 2, etc.). Although the steam generating apparatus 40 of the present embodiment is provided inside the personal care room 10 (see FIG. 1, etc.), the steam generating apparatus 40 may alternatively be provided outside the personal care room 10, as long as it can supply steam containing an herbal medicine component into the personal care room 10. Although the arrangement of the steam generating apparatus 40 inside the personal care room 10 is not particularly limited, in the present embodiment, for example, the steam generating apparatus 40 is arranged in the deepest part of the personal care room 10 (the farthermost position from the entrance/exit 14), which is located on the side of the user's head when he/she is in a supine or prone position on the bedrock (see FIG. 1, etc.). In addition, in the present embodiment, a covering plate 44 is arranged at a position where such covering plate 44 covers at least part of the user's head when he/she is in a supine or prone position on the floor 12 (see FIG. 2, etc.). The covering plate 44 functions as a cover that prevents the steam generated from the steam generating apparatus 40 from falling directly on the user's head.

The pot 41 is used for boiling an herbal medicine by using the heater 42, and the pot 41 is heated with water and the herbal medicine placed therein. Accordingly, any container may be used as the pot 41 in the steam generating apparatus 40, as long as it has a predetermined capacity, heat resistance and stability. However, if the pot 41 is configured not only to simply boil an herbal medicine, but also to generate far-infrared radiation at the same time, it is possible to expect synergistic effects of the drug efficacy of the herbal medicine and the effect of the far-infrared radiation. A charcoal pot containing charcoal as a raw material may be used as the pot 41, and the use of such charcoal pot can be expected to provide various effects of charcoal, more specifically, an effect of suppressing cell senescence in a human body, an effect of maintaining blood and body fluid in an alkaline state, an effect of releasing negative ions, an effect of supplying natural minerals, an effect of blocking electromagnetic waves, an effect of fining water molecules in the steam so as to enhance purification power for cells, and so on. When the herbal medicine has a salt content, by boiling such salt content together with the herbal medicine in the pot 41, it is possible to expect the synergistic effect of the drug efficacy of the herbal medicine, the effect of the far-infrared radiation, and the effect of the mineral components. Although not particularly shown in the drawings, it is preferable to prepare a charcoal plate surrounding such pot 41. When the pot 41 is surrounded by such charcoal plate, it is possible to expect that the effect of such charcoal plate will also be exerted in addition to the above-described effects of the charcoal pot.

The heater 42 is a tool for heating the pot 41. Although the present embodiments employ an induction heater (IH) which heats the pot 41 from its bottom, any kind of tools may be used, as long as such tool has a predetermined output and safety. In addition, from the viewpoint of preventing overheating, a tool comprising a dry-heating prevention mechanism or a timer is also preferable.

Examples of the herbal medicine (herb) that is used in the steam generating apparatus 40 may include medicinal herbs having a drug efficacy or an aroma. Preferred specific examples of such medicinal herbs may include, but are not particularly limited to, mugwort having an effect of prompting sweating, and further include Japanese angelica root, cnidium rhizome, citrus unshiu peel, clove, artemisia leaf, chrysanthemum indicum, star anise, immature orange, angelica dahurica root, artemisia capillaris flower, mentha herb, saurus chinensis, ephedra herb. and so on, and an original herb may be formulated by selecting and blending part or all of such multiple types of medicinal herbs.

When the herb formulated by blending all or part of the above-described medicinal herbs is used in the personal care apparatus 1, the synergistic drug effect of the respective medicinal herbs is exerted so as to appropriately heat the user's body and activate the user's metabolism. It is preferable for the herb to be formulated so as to be mild enough to moderately accelerate a user's metabolism that is not too active, so that many users can generally use such herb.

In this way, the herb in the present embodiment is blended and formulated so as to be suitable for many users; however, assuming that it may not be suitable for some users to have their bodies heated, another package of herbs may be prepared so that such users can feel comfortable and be satisfied with the use of such herbs. When making such package, the medicinal herbs may be pulverized and packed in a bag for use.

The air blowing apparatus 50 is an apparatus provided in the personal care room 10 in order to circulate the steam. Although the arrangement of the air blowing apparatus 50 is not particularly limited, it is preferable for the air blowing apparatus 50 to be arranged close to, and above or beside, the steam generating apparatus 40, from the viewpoint of efficiently convecting and circulating the steam (see FIG. 1, etc.). By using such air blowing apparatus 50 to first send the steam upward and then cause the steam to be circulated inside the personal care room 10, it is possible to easily send the steam from above toward the whole body of the user who is in a supine or prone position. It should be noted that the air blowing apparatus 50 may utilize the flow of air that is sent from a temperature conditioning apparatus (not shown) toward around the head of a user who is in a supine or prone position, instead of using the air blower provided in the personal care room 10, which is shown in the drawings.

The control apparatus 70 is an apparatus that controls the personal care apparatus 1. Although not shown in detail, the control apparatus 70 is constituted by a sensor that detects a temperature and a humidity, and a conditioner that provides feedback control for the temperature and humidity based on the detected values of the sensor. The control apparatus 70 of the present embodiment controls each of the room temperature and the humidity of the personal care room 10, as well as the surface temperature of the bedrock 16RP, so that a comfortable room environment can be provided.

### Control and Provision of Room Environment by the Personal Care Apparatus

The personal care apparatus 1 of the present embodiment is configured to use the above-described control apparatus 70 to control the temperature and the humidity of the personal care room 10, as well as the surface temperature of the bedrock 16RP, and to provide a room environment where all of the temperature and the humidity of the personal care room 10 and the surface temperature of the bedrock 16RP are regulated so as to be within their respective predetermined ranges. For example, the predetermined ranges herein may be as follows.
Room temperature inside the personal care room 10: 35-40° C.
Humidity inside the personal care room 10: 60-70%
Surface temperature of the bedrock 16RP: 38-43° C.
The more preferred range for the humidity is around 70%. Such preferred ranges can be achieved by performing control by aiming at the following values as the respective target values (i.e., optimum values).
Room temperature inside the personal care room 10: 45° C.
Humidity inside the personal care room 10: 70%
Surface temperature of the bedrock 16RP: 60° C.

The above-described target values or predetermined ranges are based on the new finding that, by creating the effect of accelerating metabolism through approaches from three aspects, including the room temperature, the humidity and the far-infrared radiation heat, it is possible to provide a level of comfort that the users have never experienced before, and in easier and more casual ways than before. Furthermore, by utilizing the far-infrared radiation heat while regulating the environment inside the personal care room 10 on the basis of the above-described target values or predetermined ranges, it is possible to achieve particular effects that could not be achieved by the conventional personal care apparatuses, such as the effect of improving the users' physical sensation and the effects to higher levels, and the effect of enabling a thermal approach to the deep parts of the body due to the unique effect of the far-infrared radiation heat, thereby accelerating the activation of users' metabolism.

Furthermore, in the personal care apparatus 1 of the present embodiment, the steam containing an herbal medicine component is supplied in a state in which the user's body has been heated and the activation of his/her metabolism has been accelerated, which can create an environment where the herbal medicine component can be easily taken into the user's body through his/her respiratory organs and skin without causing the user to feel pain. In addition, when the user is in a supine or prone position, the grounding area (contacting area) with his/her body is large; therefore, the steaming effect can be obtained with the user's body being in a heated condition with his/her deep body temperature having been increased throughout the body, enabling the further enhancement of the metabolizing effect and the drug efficacy.

The personal care apparatus 1 of the present embodiment can be applied not only to an apparatus for a private room which is used by a single user (alone), as has been described above, but also to an apparatus for a room which multiple users occupy at the same time to care for their bodies or engage in group exercise without worrying about others. Furthermore, considering that the configuration of the apparatus is relatively simple, there is an advantage in that the personal care apparatus 1 can be constructed by additionally introducing it in existing personal care apparatuses or existing care facilities.

### Invitation to Exercise by the Personal Care Apparatus, and Provision Thereof

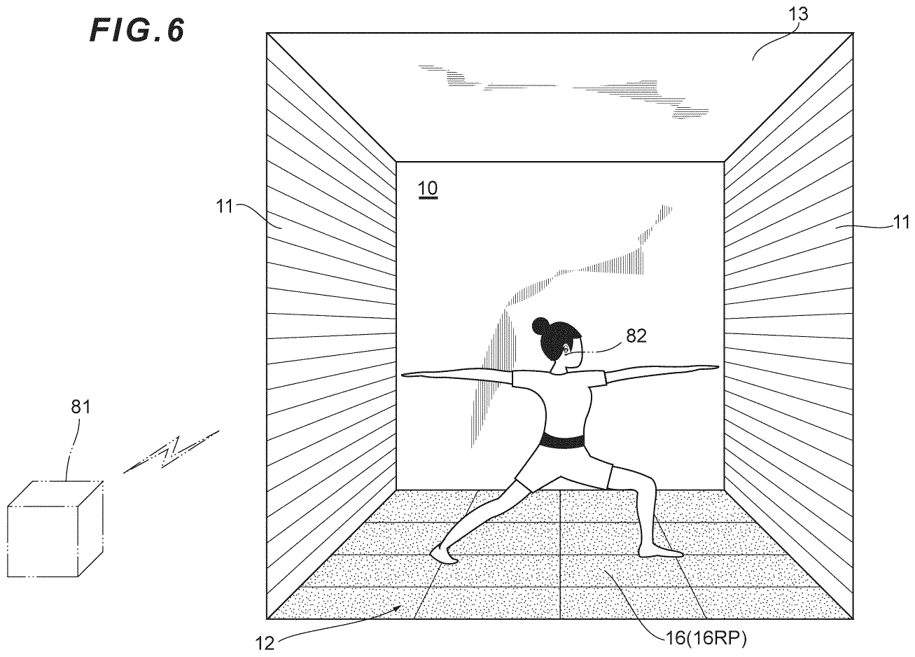

The personal care apparatus 1 of the present embodiment further comprises: an audio signal transmission system 81 that transmits an audio signal for the user; and an audio output system 82 that outputs sound based on the audio signal transmitted from the audio signal transmission system 81 (see FIG. 6). As has already been described, according to the personal care apparatus 1 of the present embodiment, the user can not only easily enjoy the effect of far-infrared radiation heat while relaxing by lying down or sitting on the floor while engaging in meditation or just relaxing, but can also obviously obtain a synergistic effect by engaging in a mild workout or gymnastics (exercise programs) such as stretching, yoga, Pilates, or various types of exercises in the personal care room 10 while enjoying the-above-described effect of the far-infrared radiation heat. When the user engages in a mild workout or gymnastics, the audio signal transmission system 81 and the audio output system 82 can not only provide the user with healing music and the like, but also play sound for prompting a light workout or gymnastics (for example, a pre-recorded guide sound that serves as a guidance for workout or gymnastics) to thereby encourage a user to engage in a workout or gymnastics based on his/her own motivation, or utilize such sound as an invitation to engage in a workout or gymnastics, which makes it possible for the user to engage in so-called “listening training.” Such configuration makes it possible to help a user to effectively increase his/her deep body temperature. The audio output system 82 may utilize an apparatus, such as a speaker, that is installed in the personal care room 10, or utilize a wearable receiver such as a wireless earphone. The ceiling 13 of the personal care room 10 may be set to a height which a standing user can reach (or can almost reach) by extending his/her arms (e.g., 1800 mm or more, more specifically, about 2300 mm). It can be said that: such personal care room 10 allows the user to experience an appropriate space which is not too wide or too small for engaging in exercise, etc., and has the ceiling 13 that has a height suitable for installing handrails or tools in such personal care room 10.

Although the embodiment described above is merely one example of a preferred embodiment, the present invention is not limited thereto and various modifications may be made without departing from the gist of the present invention.

### Industrial Applicability

The present invention is suited to be applied to various types of personal care apparatuses which provide effects of alleviating fatigue and stress, and relaxing and healing users, and thereby care for the physical and mental conditions of the users.

### REFERENCE SIGNS LIST

1: personal care apparatus
10: personal care room
11: wall
12: floor
13: ceiling
14: entrance/exit
15: door
16: interior material
16RP: bedrock (interior material containing ores)
17: partitioning wall
30: heating apparatus
31: supporting member
31a: front surface
31b: recess
31c: lid
31d: bottom surface
32: heater
32a: front surface
32b: bottom surface
33: heat insulating material
33a: front surface
33b: bottom surface
34: holding member
34a: front surface
35: temperature sensor
36: temperature regulating part
37: aluminum foil member (metal film member)
38: stainless-steel panel (metal panel member)
40: steam generating apparatus
41: pot
42: heater
44: covering plate
50: air blowing apparatus
60: indirect lighting apparatus
70: control apparatus
81: audio signal transmission system
82: wireless earphones (audio output system)

1. A personal care apparatus constituting a personal care room that can be used to increase a deep body temperature of a user, the apparatus comprising:
a bedrock installed on a floor such that a proportion of the bedrock occupying a surface area inside the personal care room is 10% or more, the bedrock containing lava that has air permeability and generates far-infrared radiation;
an electric heater that heats the bedrock from an inside or a backside thereof;
a plate-shaped heat insulating material arranged so as to support a bottom surface of the electric heater from below;
a plate-shaped holding member arranged so as to support a back surface of the heat insulating material;
a metal film member provided at least on an upper surface of the electric heater;
a temperature sensor mounted in a recess formed in a front surface of the bedrock;
a temperature regulating part that regulates a magnitude of electric power to be supplied to the electric heater so as to regulate an amount of heat generated by the electric heater;
a control apparatus that controls a surface temperature of the bedrock to be 38-43° C.;
an audio signal transmission system that transmits an audio signal to the user; and
an audio output system that outputs sound based on the audio signal transmitted from the audio signal transmission system,
wherein each of the bedrock, the heater and the heat insulating material is simply placed, without being fixed, on an upper side of the holding member.

2. The personal care apparatus according to claim 1, further comprising a metal panel member arranged between the metal film member and the bedrock.

3. The personal care apparatus according to claim 1, wherein the audio output system comprises wireless earphones worn by the user.

4. The personal care apparatus according to claim 1, wherein the bedrock installed on the floor has a size and a shape that are sufficient for the user to take a supine or prone position.

5. The personal care apparatus according to claim 1, further comprising a steam generating apparatus that supplies steam inside the personal care room.

6. The personal care apparatus according to claim 5, wherein the steam generating apparatus boils water or hot water with herbal medicine therein and supplies steam containing an herbal medicine component.

7. The personal care apparatus according to claim 6, wherein a medicinal herb having a drug efficacy or aroma is used as the herbal medicine.

8. The personal care apparatus according to claim 7, wherein the medicinal herb has been pulverized and packed in a bag.

9. The personal care apparatus according to claim 6, wherein the steam generating apparatus is provided in the personal care room.

10. The personal care apparatus according to claim 9, further comprising an air blowing apparatus that blows air so as to cause the steam containing the herbal medicine component supplied from the steam generating apparatus to move toward an upper portion of the room and then cause the steam to move downward from the upper portion of the room due to air circulation inside the room.

11. The personal care apparatus according to claim 1, wherein the control apparatus controls a room temperature inside the personal care room to be 35-40° C. and controls a humidity to be 60% or higher.

12. A personal care room comprising the personal care apparatus according to claim 1.

13. The personal care room according to claim 12, formed in a size and a shape intended for a single user.

14. The personal care room according to claim 12, formed in a size and a shape sufficient for one or multiple users to occupy and engage in exercise therein.
